(19) United States
(12) Patent Application Publication
(10) Pub. No.: US 2024/0397915 A1
(43) Pub. Date: Dec. 5, 2024

(54) ECOLOGICAL MANAGEMENT METHOD AND SYSTEM FOR TROPICAL MARINE RANCHING, DEVICE, AND MEDIUM

(71) Applicants: Hainan University, Haikou (CN); Hainan Qingxiao Environmental Testing Co., Ltd., Sanya (CN); Hainan Qianchao Ecological Technology Co., Ltd., Sanya (CN); Shenzhen Academy of Environmental Sciences, Shenzhen (CN)

(72) Inventors: Sai Wang, Haikou (CN); Tuantuan Wang, Haikou (CN); Zitan Qin, Sanya (CN); Yinglong Liu, Sanya (CN); Yuxi Yang, Sanya (CN); Dingying Wang, Sanya (CN); Enni Wu, Haikou (CN); Wangqing Tang, Haikou (CN); Nanlin Chen, Haikou (CN); Gong Cheng, Shenzhen (CN); Donghai Wu, Haikou (CN); Yongduo Song, Haikou (CN); Hongjin Zhang, Haikou (CN); Shuaihao Ding, Haikou (CN); Rongmei Zi, Sanya (CN); Jialin Yang, Sanya (CN)

(21) Appl. No.: 18/346,883

(22) Filed: Jul. 5, 2023

(30) Foreign Application Priority Data
Jun. 1, 2023 (CN) 2023106566553

(51) Int. Cl. A01K 61/13; A01K 29/00; G06Q 10/0633; G06Q 10/0637; G06Q 50/02

(52) U.S. Cl. CPC A01K 61/13; A01K 29/005; G06Q 10/0633; G06Q 10/0637; G06Q 50/02

(57) ABSTRACT

An ecological management method and system for tropical marine ranching, a device, and a medium are provided, which relates to fishery resource management technologies. The method includes: obtaining ecological environment data and energy flow analysis data of a target marine ranching; performing ecological evaluation on the ecological environment data to determine an ecological comprehensive evaluation result, the ecological comprehensive evaluation result includes a health condition evaluation and an ecosystem stability evaluation of the tropical marine ranching; performing, based on the energy flow analysis data and the ecological comprehensive evaluation result, bio-capacity evaluation by using an Ecopath energy flow model to obtain a current bio-capacity; and performing ecological management and developing an ecological management strategy based on the current bio-capacity, the ecological management strategy includes an enhancement and releasing scheme and a fishing scheme. The method can obtain a unified evaluation standard and facilitate ecological management of marine ranching.

### TECHNICAL FIELD

The disclosure relates to the field of fishery resource management technologies, and more particularly to an ecological management method and system for tropical marine ranching, a device, and a medium.

### BACKGROUND

Under influence of increasing fishing intensity, fishery resources suffer pressure of overfishing, and sustainable utilization of resources faces challenges. With rapid growth of fishery biological breeding and breeding technology, humans have already could perform a large-scale artificial intervention on natural ecology by using artificial mediation methods such as enhancement and release, that is construction of marine ranching.

The marine ranching is a small-scale semi artificial ecosystem constructed under the artificial intervention, in which the large-scale artificial intervention on natural ecology is performed to obtain continuous output of biological resources by using methods such as enhancement and release, and building an artificial habitat. Unfortunately, an evaluation technique for resource sustainability and ecological effects applicable to the marine ranching is lacked at present, and a model of resource management and utilization is often established based on experience, which results in instability of resource output and economic benefit, and a significant potential ecological risk.

Thus, resource evaluation models in related art still have problems such as inconsistent application spatial scale, mainly targeting single species and lacking nutrient interactions among multiple species, and a tropical marine ranching cannot be effectively managed.

### SUMMARY

A purpose of the disclosure is to provide an ecological management method for tropical marine ranching and an ecological management system for tropical marine ranching, a device, and a medium, so as to obtain a unified evaluation standard and facilitate ecological management of the tropical marine ranching.

In order to achieve the above purpose, the disclosure provides the following solutions:

An ecological management method for tropical marine ranching is provided, and the method includes: obtaining ecological environment data and energy flow analysis data of a target marine ranching; performing ecological evaluation on the ecological environment data to determine an ecological comprehensive evaluation result, the ecological comprehensive evaluation result including a health condition evaluation and an ecosystem stability evaluation of the tropical marine ranching; performing, based on the energy flow analysis data and the ecological comprehensive evaluation result, bio-capacity evaluation by using an Ecopath energy flow model to obtain a current bio-capacity; and performing ecological management and developing an ecological management strategy based on the current bio-capacity, the ecological management strategy including an enhancement and release scheme and a fishing schemes.

In an embodiment, the ecological environment data includes seawater quality, marine sediments, plankton, fishery resources, benthos and biological attachment of artificial reefs; and the energy flow analysis data includes biological resource survey data and consumer bio-food data.

In an embodiment, the performing ecological evaluation for the ecological environment data to determine an ecological comprehensive evaluation result, includes: performing health evaluation on the ecological environment data by using a biological community indicator method to determine the health condition evaluation of the target marine ranching, where the biological community indicator method includes a AZTI's marine biotic index (AMBI) method and an index of biotic integrity (IBI) method; and performing a stability evaluation on the ecological environment data by using an ecosystem model method to determine the ecosystem stability evaluation of the target marine ranching.

In an embodiment, the performing, based on the energy flow analysis data and the ecological comprehensive evaluation result, bio-capacity evaluation by using an Ecopath energy flow model to obtain a current bio-capacity, includes: determining a keystone species and a target species of the target marine ranching; and performing, based on the energy flow analysis data, the bio-capacity evaluation on each of the keystone species and the target species by using the Ecopath energy flow model, to obtain the current bio-capacity of each of the keystone species and the target species.

In an embodiment, performing ecological management and developing an ecological management strategy based on the current bio-capacity, includes: estimating a maximum enhancement and release intensity and a maximum sustainable yield of the target marine ranching based on the current bio-capacity, and developing the ecological management strategy based on the maximum enhancement and release intensity and the maximum sustainable yield.

The disclosure provides an ecological management system for tropical marine ranching, includes a data acquisition module, an ecological evaluation module, a bio-capacity determination module, and a strategy development module.

The data acquisition module is configured to obtain ecological environment data and energy flow analysis data of a target marine ranching.

The ecological evaluation module is configured to perform ecological evaluation on the ecological environment data to determine an ecological comprehensive evaluation result; and the ecological comprehensive evaluation result includes a health condition evaluation and an ecosystem stability evaluation of the tropical marine ranching.

The bio-capacity determination module is configured to perform, based on the energy flow analysis data and the ecological comprehensive evaluation result, bio-capacity evaluation by using an Ecopath energy flow model to obtain a current bio-capacity.

The strategy development module is configured to perform ecological management and develop an ecological management strategy based on the current bio-capacity; and the ecological management strategy includes an enhancement and release scheme and a fishing scheme.

The disclosure provides an electronic device, includes a memory and a processor, the memory is stored with a computer program, and the computer program is configured to cause, when being executed by the processor, to make the electronic device to implement the above ecological management method for tropical marine ranching.

The disclosure provides a non-transitory computer-readable storage medium having a computer program stored therein, and the computer program is configured to be executed by the processor to implement the above ecological management method for tropical marine ranching.

Based on embodiments provided in the disclosure, the disclosure provides the following beneficial effects.

The disclosure provides an ecological management method for tropical marine ranching and a system thereof, a device, and a medium, the method includes obtaining ecological environment data and energy flow analysis data of a target marine ranching to obtain an unified evaluation standard, and performing ecological evaluation and bio-capacity evaluation to obtain evaluation results, and developing an ecological management strategy based on the evaluation results, so as to facilitate ecological management of marine ranching, and achieve sustainable use of the marine ranching.

### BRIEF DESCRIPTION OF DRAWINGS

In order to provide a clearer explanation of technical solutions in embodiments of the disclosure or related art, then drawings required in the embodiments will be briefly introduced below. It is apparent that the drawings described below are merely some embodiments of the disclosure, for those skilled in the art, other drawings can also be obtained based on the described drawings without sacrificing creative works.

FIG. 1 illustrates a flowchart of an ecological management method for tropical marine ranching based on an embodiment of the disclosure.

FIG. 2 illustrates a schematic diagram of a resource adaptive management scheme of marine ranching based on an embodiment of the disclosure.

FIG. 3 illustrates a structural block diagram of an ecological management system for tropical marine ranching based on an embodiment of the disclosure.

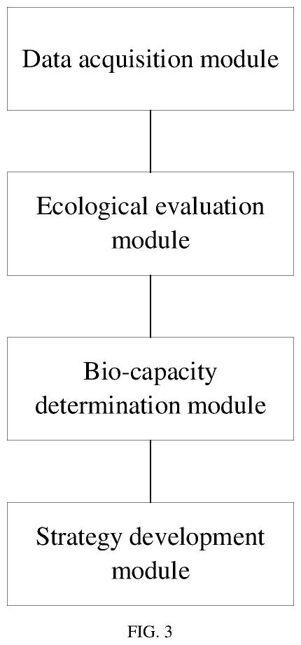

### DETAILED DESCRIPTION OF EMBODIMENTS

Technical solutions in embodiments of the disclosure will be clearly and completely described in conjunction with drawings in the embodiments of the disclosure in the follows. Obviously, the described embodiments are merely some embodiments of the disclosure, not all of them. Based on the embodiments of the disclosure, all other embodiments obtained by those skilled in the art without creative works fall within the scope of protection of the disclosure.

A purpose of the disclosure is to provide an ecological management method for tropical marine ranching and an ecological management system for tropical marine ranching, a device, and a medium, so as to obtain a unified evaluation standard and facilitate ecological management of marine ranching.

In order to make the above purpose, features, and advantages of the disclosure to be more obvious and understandable, the following provides a further detail description for the disclosure in conjunction with the drawings and the embodiments.

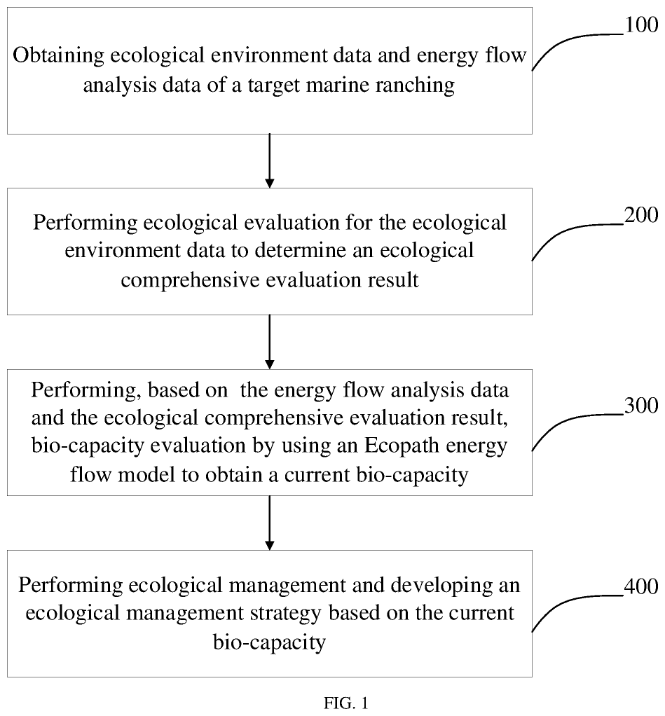

As shown in FIG. 1, the disclosure provides an ecological management method for tropical marine ranching, includes the following steps 100 to 400.

In step 100, ecological environment data and energy flow analysis data of a target marine ranching are obtained. The ecological environment data includes seawater quality, marine sediments, plankton, fishery resources, benthos and biological attachment of artificial reefs; the energy flow analysis data includes biological resource survey data and consumer bio-food data.

In step 200, an ecological comprehensive evaluation result is determined by performing ecological evaluation on the ecological environment data; and the ecological comprehensive evaluation result includes a health condition evaluation and an ecosystem stability evaluation of the tropical marine ranching.

Specifically, in step 200, health evaluation is performed on the ecological environment data by using a biological community indicator method to determine the health condition evaluation of the target marine ranching; the biological community indicator method includes a AZTI's marine biotic index (AMBI) method and an index of biotic integrity (IBI) method; and a stability evaluation of the ecological environment data is performed by using an ecosystem model method to determine the ecosystem stability evaluation of the target marine ranching. An evaluation result is shown in Table 1.

Specifically, a calculation formula of the AMBI index method is as follows:

$$\\ \begin{matrix}AMBI = ( 0. \times {GI}^{{}_{} ′} + 1.5 \times {GII}^{{}_{} ′} + 3. \times {GIII}^{{}_{} ′} + 4.5 \times {GIV}^{{}_{} ′} + 6. \times {GV}^{{}_{} ′} ) / 100 & ( 1 )\end{matrix}$$

In the formula (1), GI′ is a relative abundance of a disturbance-sensitive organism; GII′ is a relative abundance of a disturbance-insensitive (also referred to as disturbance-indifferent) organism; GIII′ is a relative abundance of a disturbance-tolerant organism (e.g., a tubicolous polychaeta spionidae species); GIV′ is a relative abundance of a second order opportunistic species type organism (e.g., a small spionidae species); GV′ is a relative abundance of a first order opportunistic species type organism (e.g., a swallowing deposit-feeding species).

TABLE 1
Health condition of benthic community
corresponding to AMBI index scores

| | Dominant | | | |
|---|---|---|---|---|
| | biological | | Station | Ecological |
| AMBI | community | Heath of benthic | disturbance | quality |
| index | group | community | level | condition |
| 0-0.2 | I | Normalcy | Undisturbed | Advanced |
| 0.2-1.2 | II | Weakness | Slightly | excellent |
| 1.2-3.3 | III | Unbalance | disturbance | |
| 3.3-4.3 | IV | Slightly polluted | Moderately | Middle |
| 4.3-5.0 | | Moderately | disturbance | Unhealthy |
| | | Polluted | | |
| 5.0-6.0 | V | Heavy pollution | Heavy | Extremely |
| | | | disturbance | unhealthy |
| 6.0-7.0 | Inanimate | Inanimate | Extreme | |
| | | | disturbance | |

An evaluation system of the IBI index method is established by analyzing a species composition, a diversity and a functional structure of a fish community and a benthic community. Indicator values vary depending on time series and a site (region), which contributes to evaluate an integrity of the studied biological community from perspectives of time and space.

An assignment method of 1, 3, and 5 is used to score biological indicators, for indicators that decrease with increasing disturbance, an actual value of each indicator is evenly divided into three intervals from lowest to highest range, a score of the indicator in a high value interval is marked as 5 points, a score of the indicator in a median interval is marked as 3 points, and a score of the indicator in a low value interval is marked as 1 point.

Scores of all of the indicators are added to obtain an index value of IBI. An added score is divided into 5 equal parts to obtain classification criteria for 5 levels of health (38˜40 points), suboptimal health (28˜32 points), general (19˜23 points), poor (8˜12 points) and extremely poor (0 point).

In step 300, bio-capacity evaluation is performed based on the energy flow analysis data to obtain a current bio-capacity by using an Ecopath energy flow model.

Specifically, in step 300, a keystone species and a target species of the target marine ranching are determined; and the bio-capacity evaluation is performed on each of the keystone species and the target species by using the Ecopath energy flow model based on the energy flow analysis data to obtain the current bio-capacity of each species.

Specifically, the Ecopath energy flow model is established based on an Ecopath model, and the Ecopath model is a mass balance model based on an ecosystem method. Based on thermal mechanical theory, a balance is maintained between the input and output of substances or energy for a certain biological function group in an ecosystem, that is, productivity is a sum of a predation mortality, a self-accumulation of organisms, a fishing catch, a migration volume, and other mortality. A formula of the Ecopath energy flow model is as follows.

$$\\ \begin{matrix}{B}_{i} \times {( \frac{P}{B} )}_{i} - {\sum }_{j} ( {B}_{j} \times {( \frac{Q}{B} )}_{j} \times {DC}_{ij} ) - {Y}_{i} - {BA}_{i} - {E}_{i} - {B}_{i} \times {( \frac{P}{B} )}_{i} \times ( 1 - {EE}_{i} ) = 0 & ( 2 )\end{matrix}$$

In the formula (2), Bᵢ is a biomass (represented by energy, dry weight, wet weight, nutrients such as carbon abbreviated as C, and nitrogen abbreviated as N) of a function group (i); (P/B)ᵢ is a ratio of productivity to the biomass of the function group (i); (Q/B)ᵢ is a ratio of total food intake to the biomass of the function group (i); EEᵢ is an ecotrophic efficiency of the function group (i); diet matrix (DC) is a n×m food composition matrix (n is an amount of predators, and m is an amount of preys), and DC is used for showing a predation-prey relationship of different function groups of organisms in an ecosystem; DCⱼᵢ is a proportion of a prey (j) in a food composition of a predator (i); BAᵢ is a biological accumulation volume of the function group (i); Yᵢ is a fishing catch or a harvest volume of the function group (i); and Eᵢ is a net migration (a moving out volume minus a moving in volume).

In step 400, ecological management is performed and an ecological management strategy is developed based on the current bio-capacity; and the ecological management strategy includes an enhancement and release scheme, and a fishing scheme.

Specifically, in the step 400, a maximum enhancement and release intensity and a maximum sustainable yield (MSY) of the target marine ranching are estimated based on the current bio-capacity, and the ecological management strategy is developed based on the maximum enhancement and release intensity and the maximum sustainable yield.

Specifically, during estimating a maximum enhancement and release intensity of the target marine ranching based on the bio-capacity, enhancement capacity and a survival rate of a corresponding one of the keystone species and the target species. Taking the target species for an example, difference between the bio-capacity and a standing biomass of the target species for enhancement is taken as the enhancement capacity of the target species for enhancement. The maximum enhancement and release intensity of the target species for enhancement is estimated by obtaining a total mortality rate and a target weight of the target species for enhancement based on a production research data. An estimation method is as follows:

enhancement capacity=bio-capacity−standing biomass;

total enhancement capacity=enhancement capacity×area of artificial reef area;

maximum enhancement amount=total enhancement capacity÷target weight of target species;

maximum enhancement and release intensity=maximum enhancement amount of target species÷survival rate.

In an embodiment, the method further includes controlling an enhancement and release intensity and a fishing catch of each of the keystone species and the target species of the target marine ranching based on the maximum enhancement and release intensity and the maximum sustainable yield. As such, the target marine ranching can be managed effectively.

Based on the above technical solutions, a Xisha Islands marine ranching is taken as an example, the following embodiments are provided.

An ultimate goal pursued by modern marine ranching is to follow laws of the ecosystem and achieve a balance between a maximum utilization of ranching resources and sustainable development. Currently, adaptive management is mainly divided into processes such as goal setting, problem identification, scheme design and implementation, dynamic monitoring, performance evaluation, feedback and adjustment.

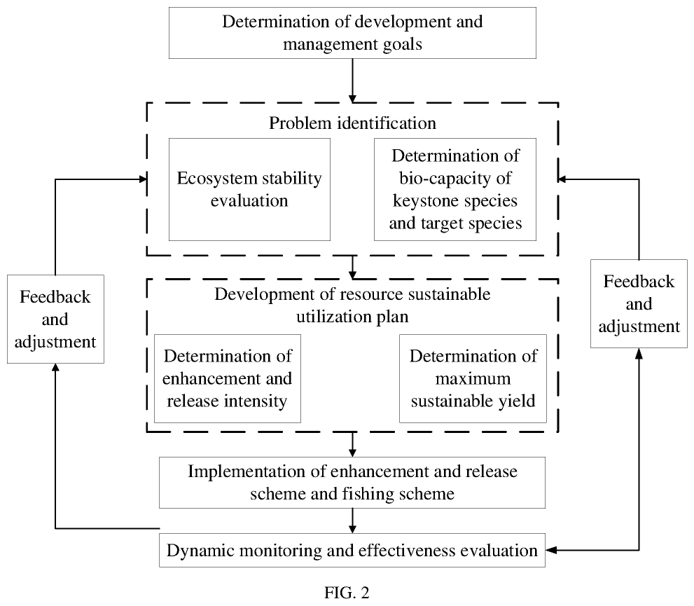

Adaptive and sustainable utilization management of marine ranching resources is to develop a dynamic adaptive management scheme for scientific enhancement and fishing of marine ranching biological resources based on the bio-capacity of the keystone species and the target species of the marine ranching under premise of maintaining health and stability of the ecosystem, through resource survey, analysis, model prediction and other technical means. The enhancement and fishing scheme is continuously adjusted based on implementation and monitoring, evaluation and feedback of the scheme to implement dynamic management of the ranching resources. During the spiral upward cycle, a technical system related to the marine ranching is continuously developed and an ability of ranching to quickly respond and adjust to uncertainty and complexity of various disturbance factors is improved, so as to implement a sustainable development goal of the ranching resources, as shown in FIG. 2.

Leisure marine ranching is primarily aimed at leisure fishing and fishing sightseeing. A resource sustainable development and management goal of the leisure marine ranching is to maintain a stability of the ecosystem and perform enhancement and conservation of ranching ecosystem keystone species, fishing and sightseeing target species based on a biological resource capacity of the marine ranching.

Thus, the ecosystem of the marine ranching is composed of a food web and habitat composed of economic organisms and key function groups. A development of the fishery resource is closely related to a quality of the habitat, the bio-capacity of the key function groups in the ecosystem, an enhancement and fishing level. Therefore, in order to identify a problem of sustainable development of the marine ranching resources, scientific investigation and mathematical model should be used to analyze, and the keystone species and the target species of the marine ranching ecosystem are determined to estimate the bio-capacity of the keystone species and the target species, so as to identify limiting factors that affect the stability of the marine ranching keystone species resources and the enhancement of the target species resources.

An ecological function type of the Xisha Islands marine ranching is a leisure fishing marine ranching. It is a marine ecological ranching featured by organic combination of tropical resource conservation and leisure tourism, with tropical reef and coral reef habitat conservation and resource enhancement as a core, integrating tropical marine tourism as a feature. Thus, a sustainable development management goal of the marine ranching resources in the Xisha Islands is to maintain the stability of the ecosystem, conserve coral reef habitats, and perform enhancement and conservation of main species of grouper and Stichopus monotuberculatus in the ranching ecosystem.

A current situation investigation and evaluation of the Xisha Islands marine ranching is as follows.

A biological resources and ecological environment research is developed and an evaluation of biological community health conditions and ecosystem stability is performed in the Xisha Islands marine ranching in July 2020 (summer), November 2020 (autumn), June 2021 (winter), and April 2021 (spring).

An evaluation result of the Xisha Islands marine ranching is as follows.

Water quality, sediment and benthos environment are good without polluted, and bio-adhesion of artificial reefs reaches more than 90%. Stabilities of the benthic community are good, and environmental quality of the community is high. Integrality of the fish community changes from good to excellent, and ecological quality changes from general to sub-health, and tends to be healthy. A utilization rate of primary productivity in sea area is less than half, which directly flows to a detrital function group, and a proportion of reuse is low. Comprehensive evaluation indicates that a CEMR value of the Xisha Islands marine ranching in Sanya is 76.22, and the stability and maturity of the ecosystem are average. The integrity of fish biological communities is good. A disturbance to the biological community is gradually decreasing, and an overall health condition of the community is improving.

An analyzation of the keystone species in the Xisha Islands marine ranching ecosystem is as follows.

Aiming at the Xisha Islands marine ranching, the Ecopath model method determines that the keystone species of the Xisha Islands marine ranching ecosystem are Cephalopholis boenack, squilla and cephalopods. A topological network model method determines that the keystone species of the Xisha Islands marine ranching ecosystem are Cephalopholis boenack, other swimming carnivorous fish (e.g., lutjanidae, trichiuridae, blenniidae, gymnothorax) and other crustaceans. The topological network model method verifies a result of the Cephalopholis boenack as the keystone species of the Xisha Islands marine ranching determined by the Ecopath model, and determines the keystone species of the Xisha Islands marine ranching is the Cephalopholis boenack. And the squilla and the cephalopods are the key function groups of the ecosystem.

The analyzation of a food web structure of the Xisha Islands marine ranching ecosystem shows that a trophic level of Epinephelus daemelii is the highest (4.08), followed by Polydactylus sextarius (3.89), a trophic level of the Cephalopholis boenack is 3.64, a trophic level of the cephalopods is 2.91, a trophic level of the squilla is 2.66, and a trophic level of Drupa is 2.56.

Determination of the target species of the Xisha Islands marine ranching is as follows.

The target species for enhancement of the leisure marine ranching should be suitable for fishing, diving, and sightseeing. The Xisha Islands marine ranching is a leisure fishing type marine ranching with combination of tropical coral reef resources conservation and tourism, main fishing species include grouper, siganids and the like. The main species for enhancement and release include grouper and Stichopus monotuberculatus. The main target species of the Xisha Islands marine ranching are determined as grouper and Stichopus monotuberculatus.

An evaluation of the bio-capacity of the Xisha Islands marine ranching is as follows.

When evaluating the bio-capacity of the marine ranching, the keystone species and the target species of the ecosystem are main evaluation objects, and some biological function groups with significant impact on the keystone species and the target species are also main evaluation objects.

The Ecopath model is used for estimating the bio-capacity of the Xisha Islands marine ranching ecosystem from a perspective of energy balance of food web, and the obtained bio-capacity of the important biological function groups is shown in Table 2.

TABLE 2
Evaluation of important economic bio-capacity
of the Xisha Islands marine ranching

| | | Standing | Capacity/ | Capacity − |
|---|---|---|---|---|
| Function | Capacity | biomass | Standing | Standing |
| groups | (t/km²/a) | (t/km²/a) | biomass | biomass |
| Grouper | 0.78 | 0.50 | 1.56 | 0.28 |
| Lutjanus | 0.45 | 0.20 | 2.25 | 0.25 |
| fulviflamma | | | | |
| Polydactylus | 0.36 | 0.12 | 3.00 | 0.24 |
| sextarius | | | | |
| Siganus | 15.70 | 1.15 | 13.59 | 14.55 |
| fuscescens | | | | |
| Portunus | 4.60 | 0.51 | 9.02 | 4.09 |
| sanguinolentus | | | | |
| stichopus | 7.10 | 0.84 | 8.47 | 6.26 |
| monotuberculatus | | | | |
| Cephalopods | 2.04 | 2.02 | 1.01 | 0.02 |
| Squilla | 1.85 | 1.29 | 1.44 | 0.56 |
| Planktonic | 22.92 | 1.74 | 13.17 | 21.18 |
| feeding fish | | | | |
| Phytophagous | 19.50 | 2.35 | 8.30 | 17.15 |
| fish | | | | |
| Sea urchins | 34.30 | 1.52 | 22.57 | 32.78 |

The results show that in the Xisha Islands marine ranching, the sea urchins, the Siganus fuscescens and the planktonic feeding fish have huge enhancement space, and their bio-capacity is 22.57 times, 13.59 times and 13.17 times of the standing biomass, respectively. However, the bio-capacity of the grouper is only 1.56 times of the standing biomass, and the enhancement space is limited.

The investigation and evaluation of the Xisha Islands marine ranching discovers that the utilization rate of the primary productivity in the sea area is less than half, and it flows directly to the detrital functional group. Sea cucumbers with detritus feeding habits have a high bio-capacity, and the bio-capacity of the sea cucumbers can reach 7.10 ton per square kilometer per year (t/km²/a), it is 8.47 times the standing biomass and has a huge potential for enhancement. This may be mainly due to its detrital feeding habits, as there is a lack of organisms in marine ranching in coral reef areas that compete directly with them for food.

In conclusion, main problems of the resource sustainable development of the Xisha Islands marine ranching are identified by the investigation and evaluation of the current situation, the determination of the keystone species and the target species, and the evaluation of the bio-capacity of the Xisha Islands marine ranching ecosystem, and the problems are as follows.

1. The stability and the maturity of the ecosystem are average. The utilization rate of the primary productivity in the sea area is less than half, and it flows directly to the detrital functional group.

2. The keystone species of the Xisha Islands marine ranching ecosystem is the Cephalopholis boenack.

3. The main target species of the Xisha Islands marine ranching are grouper and Stichopus monotuberculatus.

4. An outbreak of the Drupa and marine macroalgae effects growth and restoration of coral. In order to protect the coral reef habitat, an amount of the Drupa is controlled by moderately releasing snail predators, and the growth of the marine macroalgae is controlled by conserving the phytophagous fish (mainly Siganus).

Determination and implementation of a sustainable enhancement and release scheme, and a fishing scheme for the Xisha Islands marine ranching are as follows.

According to the comprehensive evaluation results of the biological community health condition and the ecosystem stability of the Xisha Islands marine ranching, energy flow analysis of the Ecopath model, and the analysis results of the bio-capacity, enhancement and conservation strategies are developed for different biological populations of the ranching resources, then the maximum enhancement and release intensity and the maximum sustainable yield (MSY) of the keystone species and the target species are estimated to developed the sustainable enhancement and release, and fishing schemes.

A development of the enhancement and conservation strategies of the Xisha Islands marine ranching resources is as follows.

The evaluation results of the biological community health condition of the Xisha Islands marine ranching show that the disturbance to the biological community is gradually decreasing, and the overall health condition of the community is improving. The stability of the benthic community is well, the environmental quality of the community is high, and the integrity of the fish biological community is good. Suggestions: leisure tourism activities such as sea fishing can be carried out normally, but a fishing volume in an experimental area needs to be controlled. It can maintain an existing disturbance intensity and maintain the health of the biological community.

The comprehensive evaluation results of the ecosystem stability show that the stability of the ecosystem is average, it is in the developmental stage, and the maturity is average. The utilization rate of the primary productivity in the sea area is less than half, which directly flows to the detrital function group, and the proportion of reuse is low. Suggestions: sedimentary food habits for consumers (e.g., Stichopus monotuberculatus) are added, the energy flow efficiency of the ecosystem is improved, and an ability to resist external disturbance is improved.

The analysis results of the energy flow show that benthic microalgae (4426.62 t/km²/a), planktonic bacteria (1366.49 t/km²/a), phytoplankton (1302.06 t/km²/a), coral (1182.05 t/km²/a), zooplankton (838.96 t/km²/a), sediment debris (519.46 t/km²/a), benthic bacteria (240.69 t/km²/a), other crustaceans (173.47 t/km²/a), marine macroalgae (76.73 t/km²/a), symbiotic algae (74.14 t/km²/a) and other molluscs (48.83 t/km²/a) in the ranching are not fully utilized. The Stichopus monotuberculatus and the sea urchin are main predators of the benthic microalgae, the sediment debris and the benthic bacteria, planktonic feeding fish such as Sardinella melanura is a main predator of the plankton, the Siganus fuscescens is an important predator of the marine macroalgae and the benthic microalgae, the Lutjanus fulviflamma is an important predator of the crustaceans, the Portunus sanguinolentus is an important predator of other molluscs. In general, the enhancement of the Stichopus monotuberculatus, the sea urchin, the Sardinella melanura and other plankton eating fish, the Siganus fuscescens, the Lutjanus fulviflamma, the Portunus sanguinolentus and other organisms can play a better role in improving the energy utilization efficiency of the Xisha Islands marine ranching.

The outbreak of the Drupa and the marine macroalgae effects growth and restoration of coral. In order to protect the coral reef habitat, the amount of the Drupa is controlled by moderately releasing snail predators, and the growth of the marine macroalgae is controlled by enhancing the phytophagous fish (mainly the Siganus fuscescens). Meanwhile, the Siganus fuscescens is an important species for sport fishing, the bio-capacity of the Siganus fuscescens is 13.59 times the standing biomass, and it has great potential for enhancement.

There are certain coral feeding fish in the marine ranching, the existence of the coral feeding fish poses a certain threat to the growth and repair of corals, in order to avoid increasing its biomass and damaging coral habitats, the biomass of the coral feeding fish can be controlled by enhancing grouper such as Epinephelus daemelii and the like coral feeding fish predators, so as to protect the coral habitats. Moreover, in the marine ranching, large top predators are lacked, as a larger top predator, Epinephelus daemelii can enhance and conserve this species, which can extend food chain of the ecosystem and improve structural stability of the food web of the ecosystem.

The enhancement and release intensity of the keystone species and the target species of the Xisha Islands marine ranching is as follows.

A total area of the Xisha Island marine ranching area is 10 square kilometers (km²), an area of the enhancement and release center area is 1.4 km², an area of non-enhancement and release area is 8.6 km². The Epinephelus daemelii and the Stichopus monotuberculatus are selected as the target species for enhancement, the maximum enhancement and release intensities of two target species are estimated by calculating a mortality rate of the enhancement and release species based on the evaluation results of the bio-capacity of the Ecopath model, as shown in Table 3.

TABLE 3
Maximum enhancement and release intensity of the target species of the
leisure marine ranching in the Xisha Islands marine ranching

| | | | | | | Maximum |
|---|---|---|---|---|---|---|
| | | | | | | enhancement |
| Target | Standing | Bio- | Enhanced | Target | | and release |
| species for | biomass | capacity | individual | individual | Survival | intensity (×10⁴ |
| enhancement | (t/km²/a) | (t/km²/a) | size | size (g) | rate | ind/km²) |
| Epinephelus | 0.78 | 0.50 | Body length | 207 g | 47.23% | 0.28 |
| daemelii | | | 10 cm | | | |
| Stichopus | 0.838 | 7.10 | Body length | 201 g | 30% | 10.38 |
| monotuberculatus | | | 3 cm | | | |

Based on the bio-capacity and standing biomass of the Epinephelus daemelii of the Xisha Islands marine ranching, its enhancement capacity is calculated as 0.28 t/km²/a. The target weight of the Epinephelus daemelii is 206.52 gram (g), thus, the maximum amount of the Epinephelus daemelii that can be increased is 1356 individual per square kilometers (ind/km²). An overall length of the enhancement and release of the Epinephelus daemelii is 9.63 centimeters (cm), the maximum enhancement and release intensity of the Epinephelus daemelii is 0.28×10⁴ ind/km² calculated based on the survival rate (0.4723).

Based on the bio-capacity and standing biomass of the Stichopus monotuberculatus of the Xisha Islands marine ranching, its enhancement capacity is calculated as 6.26 t/km²/a. The target weight of the Stichopus monotuberculatus is 201 g, thus, the maximum amount of the Stichopus monotuberculatus that can be increased is 31144 ind/km². An overall length of the enhancement and release of the Stichopus monotuberculatus is 3 cm, the maximum enhancement and release intensity of the Stichopus monotuberculatus is 10.38×10⁴ ind/km² calculated based on the survival rate (0.30).

A determination of the MSY of the target species of the Xisha Islands marine ranching is as follows.

A variety of mass spectrum models are used to evaluate the MSY of Epinephelus stictus and Stichopus chloronotus of the target species of the Xisha Islands marine ranching. The results show that according to current release level, fishing effort of the Epinephelus stictus can be increased by five times to the current level, and the MSY is about 0.2 t/km²/a. A fishing capacity of the Stichopus chloronotus can be doubled, the MSY is about 12 t/km²/a.

The sustainable utilization scheme of the Xisha Islands marine ranching resources is as follows:

Thus, the sustainable utilization scheme of the Xisha Islands marine ranching resources is as follows:

1. The leisure tourism activities such as sea fishing can be carried out normally, but it is necessary to control the fishing catch in the experimental area and reduce the fishing of Siganus fuscescens. It can maintain the existing disturbance intensity and maintain the health of the biological community.

2. The sedimentary food habits for consumers (e.g., Stichopus monotuberculatus), the sea urchin, the Sardinella melanura and the like planktonic feeding fish are added to improve the energy flow efficiency of the ecosystem and improve the ability to resist external disturbance. The maximum enhancement and release intensity of the Stichopus monotuberculatus is 103.8×10³ ind/km²/a.

3. In order to protect the coral reef habitat, the amount of the Drupa is controlled by moderately releasing snail predators, and the growth of the marine macroalgae is controlled by enhancing the phytophagous fish (mainly the Siganus fuscescens).

4. The biomass of the coral feeding fish can be controlled by artificial enhancement of groupers such as the Epinephelus daemelii and the like coral feeding fish predators, so as to protect the coral habitats. The maximum enhancement and release intensity of the Epinephelus daemelii is 2.83×10³ ind/km²/a.

5. According to the current release level, fishing effort of the Epinephelus stictus can be increased by five times to the current level, and MSY is about 0.2 t/km²/a. The fishing capacity of the Stichopus chloronotus can be doubled, the MSY is about 12 t/km²/a.

Dynamic monitoring, evaluation and feedback adjustment of the Xisha Islands marine ranching resources are as follows.

The biological resources and environmental factors in the Xisha Islands marine ranching are performed real-time monitoring or regular monitoring and research based on actual conditions to mastering dynamic changes in the ranching resources. Changes of the biomass of the ecosystem keystone species and target species in the ranching area such as the Cephalopholis boenack, the squilla, the cephalopods, the grouper, Stichopus japonicus, the Siganus fuscescens and the like are monitored, effects of the enhancement and release, and effects of fishing on biological populations are performed tracking performance evaluation, so as to verify the effectiveness of the developed sustainable utilization scheme for ranching resources after implementation. Effective resource evaluation, model prediction, enhancement and conservation, and management measures are tracked, summarize experience, and promote them.

Based on performance evaluation results, problems arising from the enhancement, utilization and management of the Xisha Islands marine ranching resources are reanalyze, and based on monitor data, definition of the keystone species of the ranching resources, the enhancement and release intensity of the target species, and the MSY are re-evaluated, and the sustainable utilization schemes of the ranching resources enhancement and fishing are re-optimized and re-adjusted. Time span of adjustment is no longer than 5 years, and it can be carried out once a year if conditions permit, so as to dynamically improve a response ability of the Xisha Islands marine ranching to changes in environmental factors and impacts of human activities.

The disclosure provides an ecological management system for tropical marine ranching, the system includes a data acquisition module, an ecological evaluation module, a bio-capacity determination module, and a strategy development module.

The data acquisition module is configured to obtain ecological environment data and energy flow analysis data of a target marine ranching.

The ecological evaluation module is configured to perform ecological evaluation on the ecological environment data to determine an ecological comprehensive evaluation result, and the ecological comprehensive evaluation result includes a health condition evaluation and an ecosystem stability evaluation of the tropical marine ranching.

The bio-capacity determination module is configured to perform, based on the energy flow analysis data and the ecological comprehensive evaluation result, bio-capacity evaluation by using an Ecopath energy flow model to obtain a current bio-capacity.

The strategy development module is configured to perform ecological management and develop an ecological management strategy based on the current bio-capacity; and the ecological management strategy includes an enhancement and releasing scheme and a fishing scheme.

It should be noted that, in some embodiments, the data acquisition module, the ecological evaluation module, the bio-capacity determination module, and the strategy development module described above may be implemented/embodied by one or more memories stored software modules therein and one or more processors coupled to the one or more memories and configured to execute the software modules.

The disclosure provides an electronic device, and the electronic device includes a memory and a processor, the memory is store with a computer program, and the computer program is configured to cause, when being executed by the processor, to make the electronic device to implement the above ecological management method for tropical marine ranching.

The disclosure provides a non-transitory computer-readable storage medium having a computer program stored therein, and the computer program is configured to be executed by a processor to implement the above ecological management method for tropical marine ranching.

The various embodiments in the specification are described in a progressive manner, and each embodiment focuses on differences from other embodiments. The same and similar parts between each embodiment can be referred to each other.

In the disclosure, specific embodiments are used to explain principles and implementation methods of the disclosure, and explanations of the above embodiments are merely used to help understand core ideas of the disclosure. Meanwhile, for those skilled in the art, there may be changes in specific implementation methods and application scope based on the ideas of the disclosure. In summary, content of this specification should not be understood as a limitation of the disclosure.

What is claimed is:

1. An ecological management method for tropical marine ranching, comprising:
obtaining ecological environment data and energy flow analysis data of a target marine ranching;
performing ecological evaluation on the ecological environment data to determine an ecological comprehensive evaluation result, wherein the ecological comprehensive evaluation result comprises a health condition evaluation and an ecosystem stability evaluation of the target marine ranching;
performing, based on the energy flow analysis data and the ecological comprehensive evaluation result, bio-capacity evaluation by using an Ecopath energy flow model to obtain a current bio-capacity; and
performing ecological management and developing an ecological management strategy based on the current bio-capacity, wherein the ecological management strategy comprises an enhancement and release scheme and a fishing scheme.

2. The ecological management method for tropical marine ranching as claimed in claim 1, wherein the ecological environment data comprises seawater quality, marine sediments, plankton, fishery resources, benthos and biological attachment of artificial reefs; and the energy flow analysis data comprises biological resource survey data and consumer bio-food data.

3. The ecological management method for tropical marine ranching as claimed in claim 1, wherein the performing ecological evaluation on the ecological environment data to determine an ecological comprehensive evaluation result, comprises:
performing health evaluation on the ecological environment data by using a biological community indicator method to determine the health condition evaluation of the target marine ranching, wherein the biological community indicator method comprises a AZTI's marine biotic index (AMBI) method and an index of biotic integrity (IBI) method; and
performing a stability evaluation on the ecological environment data by using an ecosystem model method to determine the ecosystem stability evaluation of the target marine ranching.

4. The ecological management method for tropical marine ranching as claimed in claim 1, wherein the performing, based on the energy flow analysis data and the ecological comprehensive evaluation result, bio-capacity evaluation by using an Ecopath energy flow model to obtain a current bio-capacity, comprises:
determining a keystone species and a target species of the target marine ranching; and
performing, based on the energy flow analysis data, the bio-capacity evaluation on each of the keystone species and the target species by using the Ecopath energy flow model, to obtain the current bio-capacity of each of the keystone species and the target species.

5. The ecological management method for tropical marine ranching as claimed in claim 1, wherein the performing ecological management and developing an ecological management strategy based on the current bio-capacity, comprises:
estimating a maximum enhancement and release intensity and a maximum sustainable yield of the target marine ranching based on the current bio-capacity, and developing the ecological management strategy based on the maximum enhancement and release intensity and the maximum sustainable yield.

6. The ecological management method for tropical marine ranching as claimed in claim 5, wherein the estimating a maximum enhancement and release intensity of the target marine ranching based on the current bio-capacity, comprises:
determining the maximum enhancement and release intensity of each of a keystone species and a target species of the target marine ranching according to an enhancement capacity and a survival rate of a corresponding one of the keystone species and the target species.

7. The ecological management method for tropical marine ranching as claimed in claim 5, the method further comprises:
controlling an enhancement and release intensity and a fishing catch of each of the keystone species and the target species of the target marine ranching based on the maximum enhancement and release intensity and the maximum sustainable yield.

8. An ecological management system for tropical marine ranching, comprising:
a data acquisition module, configured to obtain ecological environment data and energy flow analysis data of a target marine ranching;
an ecological evaluation module, configured to perform ecological evaluation on the ecological environment data to determine an ecological comprehensive evaluation result, wherein the ecological comprehensive evaluation result comprises a health condition evaluation and an ecosystem stability evaluation of the target marine ranching;
a bio-capacity determination module, configured to perform, based on the energy flow analysis data and the ecological comprehensive evaluation result, bio-capacity evaluation by using an Ecopath energy flow model to obtain a current bio-capacity; and
a strategy development module, configured to perform ecological management and develop an ecological management strategy based on the current bio-capacity, wherein the ecological management strategy comprises an enhancement and release scheme and a fishing scheme.

9. An electronic device, comprising a memory and a processor, wherein the memory is stored with a computer program, and the computer program is configured to cause, when being executed by the processor, to make the electronic device to implement the ecological management method for tropical marine ranching as claimed in claim 1.

10. A non-transitory computer-readable storage medium having a computer program stored therein, wherein the computer program is configured to be executed by a processor to implement the ecological management method for tropical marine ranching as claimed in claim 1.
